(19) United States
(12) Patent Application Publication
(10) Pub. No.: US 2018/0024147 A1
(43) Pub. Date: Jan. 25, 2018

(54) REAGENT KIT FOR DETECTING SEX HORMONE AND METHOD FOR DETECTING SEX HORMONE USING SAME

(71) Applicants: Indutry-University Cooperation Foundation Hanyang University Erica Campus, Sangrok-gu, Ansan-si (KR); Eone Reference Laboratory, Yeonsu-gu (KR)

(72) Inventors: Jaebum CHOO, Sangrok-gu, Ansan-si (KR); Hyangah CHON, Sangrok-gu, Ansan-si (KR); Rui WANG, Sangrok-gu, Ansan-si (KR); Sangyeop LEE, Sangrok-gu, Ansan-si (KR); Young Ho YOON, Yeonsu-gu (KR); Sung Hyun HONG, Gwangjin-gu (KR)

(21) Appl. No.: 15/542,432

(22) PCT Filed: Jan. 6, 2016
(86) PCT No.: PCT/KR2016/000088

§ 371 Date: Jul. 9, 2017

(30) Foreign Application Priority Data
Jan. 9, 2015 (KR) 10-2015-0003433
Jan. 5, 2016 (KR) 10-2016-0000759

(51) Int. Cl. G01N 33/74; G01N 21/65

(52) U.S. Cl. CPC G01N 33/743; G01N 21/658; G01N 2800/048

(57) ABSTRACT

The present disclosure provides a reagent kit for detecting a sex hormone, which contains a first reagent containing a metal nanoprobe in which a sex hormone and a Raman reporter are immobilized and a second reagent containing a magnetic particle in which an antibody for detecting the sex hormone is immobilized, and a method for detecting a sex hormone using the same.

### BACKGROUND

### 1. Field of Invention

The present disclosure relates to a reagent for detecting a sex hormone based on surface-enhanced Raman scattering (hereinafter, ‘SERS’), a method for detecting a sex hormone using the reagent and a method for diagnosing precocious puberty using the method.

### 2. Discussion of Related Art

On average, girls begin puberty around ages 10-11 and boys begin around ages 13-14 in Korea. And, on average, girls reach the sexual maturity level of adults at age 15 and boys at age 18, when they stop to grow.

In general, precocious puberty refers to puberty occurring before 8 years in girls or 9 years in boys. That is to say, breast development in girls before 8 years or testes development in boys before 9 years is called precocious puberty.

Recently, precocious puberty is rising meteorically as a social issue. Increased environmental hormones and childhood obesity are known as the cause of the rapid increase in patients with precocious puberty.

When the precocious puberty occurs, children may be stressed because their mental maturity does not go with physical development and their growth in height may stop as due to early closure of the growth plate. Therefore, diagnosis and/or treatment of precocious puberty is necessary.

According to a recent report by the Health Insurance Review & Assessment Service of Korea based on the analysis of the data of patients with precocious puberty, the number of Korean patients with precocious puberty has more than tripled from 21,712 in 2009 to 66,395 in 2013. Although most of the symptoms of precocious puberty can be improved with hormone therapies, accurate diagnosis is of the greatest importance. That is to say, early diagnosis of precocious puberty in children is necessary.

The representative sex hormone related with precocious puberty is estradiol. The level of estradiol in women is 60 pg/mL or higher in general, but, for men, menopausal women and girls before puberty, the level is usually 10 pg/mL or lower.

The level of testosterone is 4.04-7.21 ng/mL for adult males, 0.37-0.81 ng/mL for adult females, 2.54 ng/mL or lower for young boys and 0.20 ng/mL or lower for young girls. The level of testosterone in plasma varies 1,000-fold or more depending on age, sex and presence or absence of diseases.

The existing methods for detecting estradiol or testosterone include i) chemiluminescence-based immunoassay, ii) radioimmunoassay and iii) high-performance liquid chromatography-tandem mass spectroscopy (LC/MS/MS). The chemiluminescence-based immunoassay (ELISA) (i) is a detection method using color change and is advantages in that sensitivity is high and operation is simple. However, it has disadvantages in that detection is impossible when the concentration of estradiol (or testosterone) in the sample is low and diagnosis error occurs frequently because the limit of detection (LOD) is 30-100 pg/mL. The radioimmunoassay (ii) is a method of detecting estradiol (or testosterone) using a radioactive antibody and is advantages in that sensitivity is high with a limit of detection of 10 pg/mL. However, the accuracy is very low when the concentration of estradiol (or testosterone) in the sample is low and the sample may be contaminated by radiation. The high-performance liquid chromatography-tandem mass spectroscopy (iii) is advantages in that it is an accurate detection method. However, the detection method is complicated and requires very long time and a lot of cost. For these reasons, it is not suitable as a method for clinical diagnosis in hospitals.

At present, chemiluminescence-based automated immunoassay is widely used in most university hospitals for clinical diagnosis of precocious puberty. Representative commercialized diagnostic instruments include Abbott Architect, Beckmann, Roche Covas, Siemens ADVIA Centaur, Tosoh ST, Vitros, etc. However, the presently commercially available diagnostic instruments show large standard deviations (SD) and coefficients of variation (CV) even for an estradiol concentration range of about 50-200 pg/mL as shown in Table 1 (source: College of American Pathologists, www.cap.org/) and cannot detect estradiol at concentrations below 10 pg/mL. Accordingly, accurate diagnosis of precocious puberty is impossible with the currently available immunodiagnosis techniques and there is no gold standard method for its diagnosis.

TABLE 1
Estradiol

| | | No. | pg/mL | | | pmol/L | |
|---|---|---|---|---|---|---|---|
| | Method | Labs | Mean | S.D. | C.V. | Mean | S.D. |
| Y-01 | Abbott Architect i | 150 | 165.4 | 6.9 | 4.2 | 608.0 | 25.4 |
| | Beckman Access/2 | 54 | 284.5 | 28.6 | 10.0 | 1045.8 | 104.9 |
| | Beckman Unicel Dxl | 163 | 282.1 | 28.5 | 10.1 | 1037.2 | 104.9 |
| | Roche Cobas e411/Elecsys | 89 | 234.1 | 11.0 | 4.7 | 860.5 | 40.5 |
| | Roche Cobas e600 series/E170 | 278 | 221.0 | 8.7 | 3.9 | 812.6 | 32.0 |
| | Siemens ADVIA Centaur CP eE2 | 39 | 243.7 | 16.3 | 6.7 | 895.9 | 60.0 |
| | Siemens ADVIA Centaur/XP | 53 | 207.8 | 10.4 | 5.0 | 764.1 | 38.5 |
| | Siemens ADVIA Centaur/XP E26III | 13 | 208.2 | 8.9 | 4.3 | 765.5 | 32.6 |
| | Siemens ADVIA Centaur/XP eE2 | 221 | 208.2 | 10.0 | 4.8 | 765.4 | 36.6 |
| | Siemens Dimension Vista | 44 | 326.2 | 11.4 | 3.5 | 1199.3 | 41.9 |
| | Siemens Immulite 2000/XPi | 91 | 286.8 | 18.2 | 6.4 | 1054.5 | 67.0 |
| | Siemens Immulite/Immulite 1000 | 45 | 309.6 | 24.8 | 8.0 | 1138.1 | 91.3 |
| | Tosoh ST AIA-Pack | 32 | 590.6 | 36.8 | 6.2 | 2171.2 | 135.2 |
| | Vitros 3600, 5600, ECi, ECiQ | 103 | 366.6 | 89.6 | 24.4 | 1347.6 | 329.3 |

Non-patent document 1 (William Rosner, et al., 2013. J Clin Endocrinol Metab, 98(4) 1376-1387) describes in FIG. 1 that it is difficult to detect estradiol at concentrations below 10 pg/mL. Also, the non-patent document 1 states that accurate detection of the estradiol level at low concentrations is important for monitoring of patients with breast cancer treated with hormone inhibitors (aromatase inhibitors), because the estradiol level which is 10-15 pg/mL in general before the treatment should be maintained at 1 pg/mL or lower (see right column on p. 1379).

As described above, there is no method for accurately detecting sex hormones, e.g., estradiol or testosterone, at concentrations of 10 pg/mL or lower, at present. Also, because the concentrations of the sex hormones in children with precocious puberty should be maintained low (10 pg/mL or lower) after the hormone therapies, accurate measurement of the concentrations of the sex hormones is necessary. It is necessary to provide accurate data about the concentrations of the sex hormones of the patients for evaluation of the effect of the therapies by clinicians.

It is also emphasized that there is an urgent need for researches about an appropriated method for detecting sex hormones for diagnosis of precocious puberty in children (see right column on p. 1380 in the non-patent document 1). In addition, it is strongly asserted in the non-patent document 1 that a method capable of detecting estradiol at very low concentrations is necessary (see Conclusions on p. 1384 in the non-patent document 1).

Non-patent document 2 (Genna Rollins, May 2013, Clinical Laboratory News, Vol. 39, No. 5) published by the American Association for Clinical Chemistry stresses the limitation of the current method for diagnosing precocious puberty stating that “The current platform assays can't distinguish between 10 and 60 pg/mL.” The non-patent document 2 emphasizes the necessity of the development of a new technology that can quantify estradiol at concentrations of 10 pg/mL or lower with accuracy and reliability and can be applied for routine clinical diagnosis. As the title of the non-patent document 2, the currently needed technology is “A Call for Better Estradiol Measurement”. Specifically, the development of a new method for detecting sex hormones, which can reduce the time required for detecting the sex hormones, remarkably improve the sensitivity and accuracy of detection and, at the same, adopt the diagnostic reagent-based automated immunoassays currently used for clinical diagnosis and a detection technology for a gold standard analysis of sex hormones is urgently needed.

Surface-enhanced Raman scattering (SERS) is an analytical method capable of overcoming the limit of detection of Raman spectroscopy. This analytical method quantifies a target substance by measuring the change in the intensity of characteristic SERS peaks amplified by a Raman reporter molecule. If the reporter molecule adsorbed on a rough metal surface is exposed to an excitation source (laser light), electromagnetic and chemical enhancement occurs at the SERS active site of the reporter molecule known as a “hot junction” and the SERS signal is enhanced significantly (non-patent documents 3, 4 and 5). This enhancement effect is expected to solve the low sensitivity problem of the conventional Raman spectroscopy and is expected to overcome the accuracy and detection limit issues of the conventional chemiluminescence-based assay and radioimmunoassay.

The inventors of the present disclosure have conducted researches to overcome the above-described problems of the existing technologies. As a result, they have developed a new immunoassay method of sex hormones, which can reliably analyze sex hormones with high sensitivity using new-concept nanoplasmonics-based SERS which is different from the existing radioimmunoassay, enzyme immunoassay and chemiluminescence immunoassay used in immunoassay of sex hormones in the detection method. The present disclosure is regarded as a new-concept diagnosis technology of sex hormones capable of overcoming the problems (detection sensitivity and accuracy) of the existing diagnosis of precocious puberty through detection of sex hormones.

### REFERENCES

Non-patent document 1: William Rosner, et al., April 2013. J Clin Endocrinol Metab, 98(4) pp. 1376-1387.

Non-patent document 2: Genna Rollins, May 2013, Clinical Laboratory News, Vol. 39, No. 5.

Non-patent document 3: Kneipp, J. et al., 1997. Phys. Rev. Lett. 78, pp. 1667-1670.

Non-patent document 4: Nie, S. M. and Emory, S. R., 1997. Science 275, pp. 1102-1106.

Non-patent document 5: Kneipp, J. et al., 2006. Nano Lett. 6(10), pp. 2225-2231.

### SUMMARY OF THE INVENTION

The present disclosure is directed to providing a reagent kit for detecting a sex hormone.

The present disclosure is also directed to providing a method for detecting a sex hormone based on surface-enhanced Raman scattering (SERS).

The present disclosure is also directed to providing a method for diagnosing precocious puberty using the method for detecting a sex hormone.

In the present disclosure, a number of literatures and references are referenced and cited. The disclosures of the cited literatures and references are incorporated herein to more clearly explain the background art and the present disclosure.

The present disclosure provides a reagent kit for detecting a sex hormone, which contains

a first reagent containing a metal nanoprobe in which a sex hormone and a Raman reporter are immobilized; and

a second reagent containing a magnetic particle in which an antibody for detecting the sex hormone is immobilized.

FIG. 1 schematically illustrates the first reagent and the second reagent of the present disclosure.

The term “metal nanoprobe” or “metal nanoprobes” used in the present disclosure can be used interchangeably with a “metal nanoparticle” or “metal nanoparticles” or a “metal nanosphere” or “metal nanospheres”. The metal nanoprobe(s) refers to a metal nanostructure(s) and is a term widely used in the art.

The term “magnetic particle(s)” used in the present disclosure is can be used interchangeably with a “magnetic bead” or “magnetic beads”. The magnetic particle(s) may be made of a magnetic material(s). As the magnetic material, any one that is widely used in the art may be used. For example, one of Fe₂O₃, Fe₃O₄ or FePt may be used, although not being limited thereto.

The magnetic particle(s) and the metal nanoprobe(s) (e.g., gold nanoparticle(s)) used in the present disclosure are widely known in the art (H. Chon, et. al., Chem. Commun., 2011, 47, 12515-12517; Frens, 1973, Nature Physical Science 241, 20-22).

The Raman reporter is immobilized on the surface of the metal nanoprobe(s). The Raman reporter is capable of more effectively analyzing a target substance to be detected in SERS analysis because it exhibits a specific Raman spectrum. As the Raman reporter molecule, any one known in the art may be used. Specifically, for example, 4,4′-dipyridyl (DP), crystal violet (CV), 4-mercaptotoluene (4-MT), 3,5-dimethylbenzenethiol (3,5-DMT), thiophenol (TP), 4-aminothiophenol (4-ATP), benzenethiol (BT), 4-bromobenzenethiol (4-BBT), 2-bromobenzenethiol (2-BBT), 4-isopropylbenzenethiol (4-IBT), 2-naphthalenethiol (2-NT), 3,4-dichlorobenzenethiol (3,4-DCT), 3,5-dichlorobenzenethiol (3,5-DCT), 4-chlorobenzenethiol (4-CBT), 2-chlorobenzenethiol (2-CBT), 2-fluorobenzenethiol (2-FBT), 4-fluorobenzenethiol (4-FBT), 4-methoxybenzenethiol (4-MOBT), 3,4-dimethoxybenzenethiol (3,4-DMOBT), 2-mercaptopyrimidine (2-MPY), 2-mercapto-1-methylimidazole (2-MMI), 2-mercapto-5-methylbenzimidazole (2-MBI), 2-amino-4-(trifluoromethyl)benzenethiol (2-ATFT), benzylmercaptan (BZMT), benzyl disulfide (BZDSF), 2-amino-4-chlorobenzenethiol (2-ACBT), 3-mercaptobenzoic acid (3-MBA), 1-phenyltetrazole-5-thiol (1-PTET), 5-phenyl-1,2,3-triazole-3-thiol (5-PTRT), 2-iodoaniline (2-IAN), phenyl isothiocyanate (PITC), 4-nitrophenyl disulfide (4-NPDSF), 4-azido-2bromoacetophenone (ABAPN), X-rhodamine-5-(and-6)-isothiocyanate (XRITC), malachite green isothiocyanate (MGITC), etc. may be used, although not being necessarily limited thereto.

In an exemplary embodiment of the present disclosure, when the gold nanoparticle described above is used, malachite green isothiocyanate (MGITC) may be used as the Raman reporter. In an exemplary embodiment of the present disclosure, the Raman reporter molecule may be immobilized by adsorbing it onto the surface of the metal nanoprobe described above by mixing with the nanoprobe.

The antibody for detecting the sex hormone may include a Primary antibody and a secondary antibody, the secondary antibody may be immobilized on the magnetic particle and the primary antibody may bind to the secondary antibody and react with the sex hormone.

As the primary antibody and the secondary antibody, those commonly used in the art may be used without limitation. Specifically, the secondary antibody may be an anti-mouse antibody which is immobilized on the magnetic particle and the primary antibody may be an anti-sex hormone antibody which binds to the secondary antibody and specifically immunoreacts with the sex hormone.

In the existing immunoassay methods, a “sandwich immunocomplex” is mainly used. The “sandwich immunocomplex” refers to an immunocomplex formed from an antibody-antigen-antibody reaction. It is named so because an antigen is sandwiched between antibodies.

For sandwich immunoassay, two antibodies should be bound to a target (antigen) in a sandwich manner. However, the sex hormone (antigen) of the present disclosure is a small molecule and does not have two epitope binding sites to which two antibodies can bind in a sandwich manner of “antibody-antigen (sex hormone)-antibody”. Therefore, in the present disclosure, a competitive immunoreaction (which will be described later) is used so that assay is possible by using one epitope binding site of the sex hormone. And, when immobilizing the antibodies on the magnetic particle, the secondary antibody may be bound to primary and then the primary antibody specific for the sex hormone may be immobilized in order to increase the loading density of the antibodies.

The sex hormone may be estrogen or testosterone, although not being necessarily limited thereto.

The detectable concentration of the sex hormone may be 0.1-1,000 pg/mL. Specifically, the detectable concentration of the sex hormone may be 0.1-10 pg/mL.

When the concentration of the sex hormone is higher than 1,000 pg/mL, e.g., 3,000 pg/mL, it may be used after being diluted to 1,000 pg/mL.

The limit of detection of the sex hormone may be 0.1 pg/mL.

The detection time of the sex hormone may be 2 hours or shorter. Specifically, it may be 1-2 hours.

The estrogen may be one or more selected from a group consisting of estradiol, estrone and estriol, although not being necessarily limited thereto. The estradiol may be 17β-estradiol (E2).

The estrogen collectively refers to a female sex hormone synthesized from cholesterol in the body and secreted from the ovary, adrenal cortex, etc. and includes estradiol, estrone, estriol, etc. The estrogen binds to its receptor in the nucleus and induces female sex characteristics, thereby playing important physiological roles such as egg maturation, growth and development of the mammary gland, etc.

The estradiol is a sex hormone which is predominant in women and is the most representative estrogen. The estradiol is known to affect the change of reproductive organs such as the uterus, vagina, Fallopian tubes, testicles, etc. and the development of breasts and also affect growth disorder by inducing feminine fat distribution. Pregnant women show estradiol levels of 60 pg/mL or higher and the women who are administered with ovulation inducers have considerably high estradiol levels (250-2000 pg/mL). For men, children before puberty and menopausal women, the estradiol level is 20 pg/mL or lower. And, female breast cancer patients administered with sex hormone inhibitors show estradiol levels of 1 pg/mL or lower.

With the existing diagnostic technologies, it is difficult to detect sex hormones at 10 pg/mL or lower with high sensitivity. In order to solve this problem, the present disclosure provides a reagent which is capable of detecting a sex hormone at 10 pg/mL or lower with high sensitivity. The present disclosure also provides a reagent which can detect the sex hormone within 2 hours.

In another aspect, the present disclosure provides a SERS-based method for detecting a sex hormone, which includes the steps of:

preparing a sample solution containing a sex hormone;

preparing a metal nanoprobe in which the sex hormone is bound to a Raman reporter;

preparing a magnetic particle in which a primary antibody and a secondary antibody for detecting the sex hormone are immobilized;

adding the metal nanoprobe and the magnetic particle in which the primary antibody and the secondary antibody are immobilized to the sample solution at the same time;

forming an immunocomplex with the magnetic particle through a competitive immunoreaction of each of the sex hormone in the sample solution and the sex hormone of the metal nanoprobe with the primary antibody immobilized in the magnetic particle;

separating the magnetic particle in which the immunocomplex is formed using magnetism;

irradiating a laser light to the separated magnetic particle; and

detecting the sex hormone by measuring a surface-enhanced Raman scattering (SERS) signal after the irradiation of the laser light.

As described above, a “sandwich immunocomplex” has been mainly used. However, in the present disclosure, a “competitive immunoreaction” wherein the antibody immobilized on the magnetic particle reacts competitively with the antigen (sex hormone) bound to the metal nanoprobe and the antigen (sex hormone) present in the blood (sample solution) of a patient is used. Through this, the present disclosure reduces diagnosis time by simplifying the diagnosis procedure without forming a sandwich immunocomplex.

The competitive immunoreaction may be an immunoreaction between the sex hormone (antigen) contained in the sample solution and the magnetic particle in which the antibody specifically binding to the sex hormone is immobilized (formation of the first immunocomplex in FIG. 2) and an immunoreaction between the metal nanoprobe to which the same antigen as the sex hormone (antigen) contained in the sample solution is bound and the magnetic particle in which the antibody specifically binding to the sex hormone is immobilized (formation of the second immunocomplex in FIG. 2) occurring competitively.

The sample solution may be selected from a group consisting of a tissue extract, a cell lysate, a whole blood, a blood plasma, a blood serum, a saliva, an ocular fluid, a cerebrospinal fluid, a sweat, a urine, a milk, an ascitic fluid, a synovial fluid, a peritoneal fluid and a dried blood spot, although not being necessarily limited thereto. The dried blood spot may be may be prepared from a sample solution according to a method well known in the art. Specifically, an extract extracted from a blood blotted and dried on filer paper may be used.

The secondary antibody may be an anti-mouse antibody immobilized on the magnetic particle and the primary antibody may be an anti-sex hormone antibody which binds to the secondary antibody and specifically immunoreacts with the sex hormone.

In the step of forming the immunocomplex, the intensity of the SERS signal may be decreased as the concentration of the sex hormone in the sample solution is higher because the amount of the metal nanoprobe forming the immunocomplex with the magnetic particle is decreased; or the intensity of the SERS signal may be increased as the concentration of the sex hormone in the sample solution is lower because the amount of the metal nanoprobe forming the immunocomplex with the magnetic particle is increased.

FIG. 2 schematically illustrates a method for detecting a sex hormone according to the present disclosure.

A sample solution containing a sex hormone is prepared, a metal nanoprobe in which the sex hormone is bound to a Raman reporter is prepared and a magnetic particle in which a primary antibody and a secondary antibody for detecting the sex hormone are immobilized is prepared. The sample solution and the metal nanoprobe in which the sex hormone is bound are added to the magnetic particle in which the primary antibody and the secondary antibody are immobilized at the same time. An immunocomplex is formed by inducing a competitive immunoreaction of the sex hormone in the sample solution and the metal nanoprobe in which the Raman reporter (and the sex hormone) are bound to the primary antibody immobilized on the magnetic particle. As a result, a first immunocomplex in which the sex hormone in the sample solution and the primary antibody of the magnetic particle are bound and a second immunocomplex in which the sex hormone of the metal nanoprobe and the primary antibody of the magnetic particle are bound are formed at the same time.

After the immunocomplex is formed, the magnetic particle in which the immunocomplex is formed is separated using magnetism and a surface-enhanced Raman scattering (SERS) signal is measured by irradiating a laser light to the separated magnetic particle.

If the concentration of the sex hormone in the sample solution is high, the second immunocomplex in which the metal nanoprobe in which the Raman reporter is bound and the primary antibody of the magnetic particle are bound will be produced in a small amount. A detailed description is given referring to FIG. 4. FIG. 4 shows TEM (transmission electron microscopy) images of the immunocomplex formed through the competitive immunoreaction (The sex hormone, which is an organic material, is not observed by TEM). In FIG. 4, the numerical values on the upper left-hand corners are the concentrations of the sex hormone estradiol in the sample solutions. From FIG. 4, it can be seen that the amount of the metal nanoprobe bound to the magnetic particle is decreased as the concentration of the sex hormone in the sample solution is higher. The concentration of the metal nanoprobe used in the competitive immunoreaction of the present disclosure is optimized for testing. In an exemplary embodiment of the present disclosure, the concentration of the metal nanoprobe used is 0.12 nM and 50 μL of the metal nanoprobe is used when testing 25 μL of the sample. That is to say, the amount of the metal nanoprobe used in the competitive immunoreaction is constant. If the concentration of the sex hormone in the sample solution is high, the sex hormone in the sample solution is more likely to bind with the magnetic particle to form the immunocomplex when the sex hormone in the sample solution competes with the sex hormone of the metal nanoprobe for the competitive immunoreaction with the magnetic particle (in which the antibody is immobilized). As a result, the amount of the metal nanoprobe bound to the magnetic particle will be decreased (i.e., the amount of the second immunocomplex will be smaller than that of the first immunocomplex) and, accordingly, the measured intensity of the SERS signal will be weak. On the contrary, if the concentration of the sex hormone in the sample solution is low, the first immunocomplex of the sex hormone in the sample solution with the magnetic particle will be formed in a smaller amount. And, the amount of the second immunocomplex in which the metal nanoprobe and the primary antibody of the magnetic particle are bound will be relatively larger. In this case, the intensity of the SERS signal will be increased because the metal nanoprobe is bound to the magnetic particle in a larger amount.

Accordingly, the measured intensity of the SERS signal will be decreased as the concentration of the sex hormone in the sample solution is higher and the measured intensity of the SERS signal will be increased as the concentration of the sex hormone in the sample solution is lower.

In another aspect, the present disclosure provides a method for diagnosis of precocious puberty of a subject, which includes the steps of:

extracting a sample solution from a subject;

detecting a sex hormone for the extracted sample solution by the method for detecting a sex hormone described above; and

determining the concentration of the detected sex hormone.

The subject may be a mammal, specifically a human, more specifically a girl under 8 years of age or a boy under 9 years of age.

The concentration of the detected sex hormone may be 0.1-1000 pg/mL.

The method may further include, after the step of determining the concentration of the detected sex hormone, a step of diagnosing as precocious puberty when the concentration of the detected sex hormone is higher than 10 pg/mL.

The reagent kit for detecting a sex hormone and the method for detecting a sex hormone according to the present disclosure may be used to diagnose precocious puberty. In addition, the reagent kit and the method for detecting a sex hormone according to the present disclosure may be used for various indications related with sex hormones because even the sex hormone at very low concentrations can be detected.

For example, the present disclosure may be used for various applications such as establishment of normal estradiol levels in children depending on ages and sexes, tracking of the onset of puberty, diagnosis of precocious puberty, evaluation of therapeutic effect after treatment of precocious puberty, etc. Also, the present disclosure may be used to investigate the prognosis of therapeutic effect in breast cancer patients receiving hormone therapies (for breast cancer patients, the estradiol level should be lower than 1 pg/mL), risk factors of osteoporosis and fracture in men, diagnosis of gonadal tumors, cause of boys having female breasts, etc. The present disclosure may also be used for diagnostic and prognostic applications such as evaluation of the therapeutic effect of hormone therapies in menopausal women, investigation of the cause of hypogonadism, evaluation of the therapeutic effect of hormone therapies in prostate cancers patient, etc.

Sex hormones at very low concentrations can be detected using the reagent and method for detecting a sex hormone according to the present disclosure. Therefore, the present disclosure may be used for accurate diagnosis of precocious puberty. In addition, the present disclosure may also be used for researches on hormone-related diseases because sex hormones can be detected with high sensitivity.

### BRIEF DESCRIPTION OF THE DRAWINGS

FIG. 1 schematically illustrates a first reagent and a second reagent of the present disclosure.

FIG. 2 schematically illustrates a method for detecting a sex hormone according to the present disclosure.

FIG. 3 shows transmission electron microscopic (TEM) images (a) and dynamic light scattering (DLS) data (b) of synthesized gold nanoprobes.

FIG. 4 shows TEM images of immunocomplexes formed through a competitive immunoreaction according to the present disclosure (the numerical values on the upper left-hand corners are the concentrations of estradiol in sample solutions).

FIGS. 5a-5c show a result of measuring Raman signals using a method for detecting estradiol according to an exemplary embodiment of the present disclosure.

FIGS. 6a-6c show a result of measuring Raman signals using a method for detecting testosterone according to an exemplary embodiment of the present disclosure.

FIG. 7 shows a result of detecting estradiol by a SERS-based detection method according to an exemplary embodiment of the present disclosure and a result of detecting estradiol by ELISA analysis as a comparative example.

FIG. 8 shows a result of detecting testosterone by ELISA analysis as a comparative example.

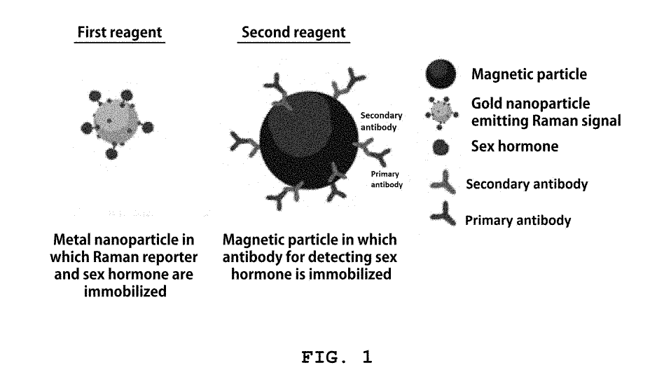

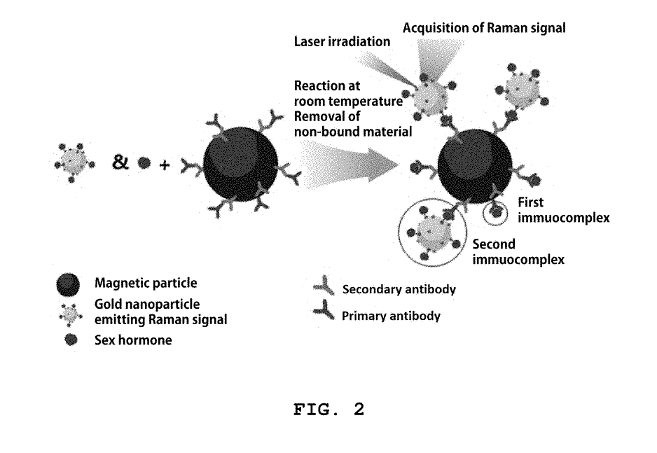

### DETAILED DESCRIPTION OF EXEMPLARY EMBODIMENTS

Hereinafter, specific examples are provided to help understanding of the present disclosure. However, the following examples only exemplify the present disclosure and it will be obvious to those of ordinary skill in the art that various changes and modifications can be made within the scope and technical idea of the present disclosure and such changes and modifications are included within the scope of the appended claims.

### EXAMPLE 1

### Preparation of First Reagent (Metal Nanoprobe)

Chloroauric acid (HAuCl₄), trisodium citrate, poly(ethylene glycol) 2-mercaptoethyl ether acetic acid (HS-PEG-COOH, MW ˜3500), poly(ethylene glycol) methyl ether thiol (HS-PEG, MW ˜2000), EDC (N-(3-dimethylaminopropyl)-N′-ethylcarbodiimide hydrochloride) and NHS (N-hydroxysuccinimide) were purchased from Sigma-Aldrich. Malachite green isothiocyanate (MGITC) was purchased from Invitrogen and estradiol-ovalbumin conjugate (E2-OVA) was purchased from Cusabio.

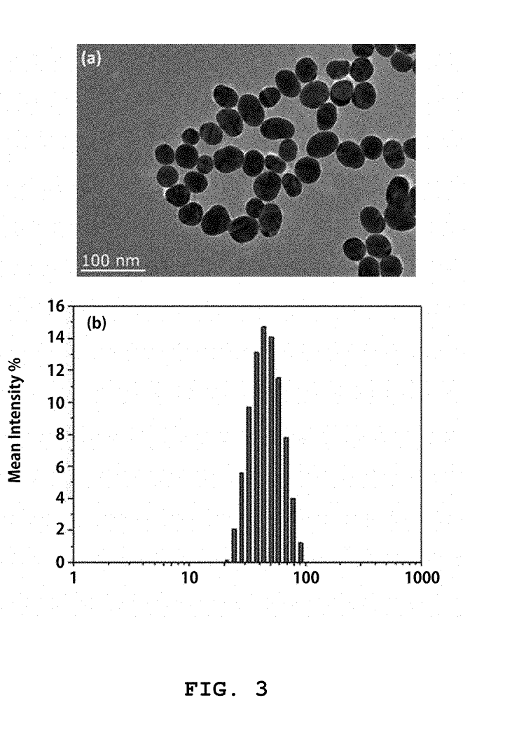

For synthesis of metal nanoprobes, spherical gold nanoparticles were synthesized (Frens, 1973, Nature Physical Science 241, 20-22.). 50 mL of a 0.01% HAuCl₄ solution was heated to boiling and 0.5 mL of a 1% trisodium citrate solution was added dropwise. At first, the color of the HAuCl₄ aqueous solution turned blue as nanoparticles (seeds) were formed. Then, the solution turned red gradually with time as the nanoparticles grew. After the color of the nanoparticles to be synthesized was confirmed, the mixture was boiled further for 15 minutes and the reaction was terminated. Then, the gold nanoparticles were aged for 4 hours or longer while cooling to room temperature. It was confirmed through transmission electron microscopy (TEM) and dynamic light scattering (DLS) measurements that gold nanoprobes were synthesized stably with uniform sizes of about 40-50 nm, as seen from FIG. 3. Subsequently, for use as a SERS substrate, the Raman reporter malachite green isothiocyanate (MGITC) was coated on the gold nanoparticles. After adding the Raman reporter to 1 mL of 0.12 nM 40-nm gold nanoparticles dropwise so that the final concentration was 50 nM and then adding 60 μL of 10 μM poly(ethylene glycol) 2-mercaptoethyl ether acetic acid (HS-PEG-COOH, MW ˜3500) and 120 μL of 10 μM poly(ethylene glycol) methyl ether thiol (HS-PEG, MW ˜2000) dropwise, the mixture was incubated for 3 hours in order to introduce carboxyl functional groups onto the surface of the gold nanoparticles. Then, estradiol-ovalbumin (E2-OVA) conjugate or testosterone-BSA (bovine serum albumin) conjugate was immobilized using the carboxyl functional groups on the surface of the gold nanoparticles. For this, after adding 5 μL of 25 mM EDC (N-(3-dimethylaminopropyl)-N′-ethylcarbodiimide hydrochloride) and NHS (N-hydroxysuccinimide) dropwise and mixing for 15 minutes, 2 μL of 1 mg/mL estradiol-ovalbumin was added dropwise and the mixture was reacted at room temperature for 2 hours. Then, the reaction mixture was incubated at 4° C. for 12 hours. Unbound residues were removed through three centrifugations (7200 rpm, 10 minutes).

### EXAMPLE 2

### Preparation of Second Reagent (Magnetic Particle)

Secondary antibodies (anti-mouse IgG (Fc-specific) antibody produced in goat), EDC (N-(3-dimethylaminopropyl)-N′-ethylcarbodiimide hydrochloride) and NHS (N-hydroxysuccinimide) were purchased from Sigma-Aldrich. Magnetic microparticles (Dynabeads MyOne™) and PBS buffer (0.1 mM, pH 7.4) were purchased from Invitrogen. Primary antibodies (anti-17β estradiol antibody or mouse anti-testosterone monoclonal antibody) were purchased from Abcam.

The secondary antibodies (anti-mouse antibody) were immobilized using the carboxyl functional groups on the surface of the magnetic microparticles. For this, the carboxyl functional groups on the surface of the magnetic microparticles were activated by adding 5 μL of 0.1 M EDC and NHS dropwise for 30 minutes. Then, after adding 2 mg/mL secondary antibodies (anti-mouse antibody) dropwise and incubating at room temperature for 2 hours, residues not bound to the surface of the magnetic microparticles were removed using magnetism. The separated magnetic particles were dissolved in PBS (10 mM, pH 7.4) and stored at 4° C. Subsequently, the preparation of magnetic particles for detecting sex hormones was completed by adding 25 μL of 0.55 g/mL anti-estradiol antibodies (or anti-testosterone antibodies) as the primary antibodies to 25 μL of the magnetic particles in which the secondary antibodies were immobilized and incubating at room temperature for 90 minutes. After the reaction, residues not bound to the surface of the magnetic microparticles were removed using magnetism and the separated magnetic particles were dissolved in PBS (10 mM, pH 7.4).

### EXAMPLE 3

### Detection of Sex Hormones Based on Surface-Enhanced Raman Scattering

3-1: Detection of Estradiol

First, blood containing estradiol was prepared as a sample solution. Then, 25 μL of the magnetic particles in which the secondary antibodies and the primary antibodies (anti-estradiol antibody) are immobilized, which was synthesized in Example 2, and 50 μL of the gold nanoprobes prepared in Example 1 were added at the same time to 25 μL of the sample solution. A total of 90 minutes was spent for the detection.

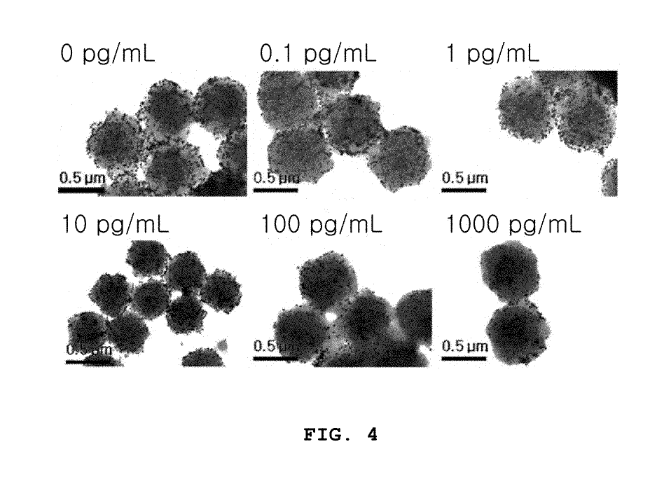

FIG. 4 shows TEM (transmission electron microscopy) images of immunocomplexes formed through a competitive immunoreaction according to the present disclosure (the numerical values on the upper left-hand corners are the concentrations of estradiol in sample solutions). As can be seen from FIG. 4, the amount of the metal nanoprobes bound to the magnetic particles decreased as the concentration of the sex hormone in the sample solution was higher.

Then, the magnetic particles in which the first and second immunocomplexes were formed were separated using magnetism and Raman analysis was conducted for the separated magnetic particles. The Raman analysis was performed as follows. The Renishaw Invia Raman spectrometer (Renishaw, UK) was used and the Spectra Physics He—Ne laser operating at 632.8 nm was used as a light source. The Rayleigh line was removed using a holographic notch filter located in the collection path. All the peak positions were calibrated by measuring the peak position of silicon as reference at 520 cm⁻¹ before measurement. Raman spectra were collected in the range of 630-1730 cm⁻¹ with an exposure time of 1 second using a laser with an output wavelength of 633 nm and an output power of 20 mW. A 20× objective lens was used to focus the laser spot. Baseline correction of all the Raman spectra was performed using the WiRE 4.0 software (Renishaw, UK). Quantitative analysis of the sex hormone was performed for the peak at 1613 cm⁻¹ which showed the strongest intensity.

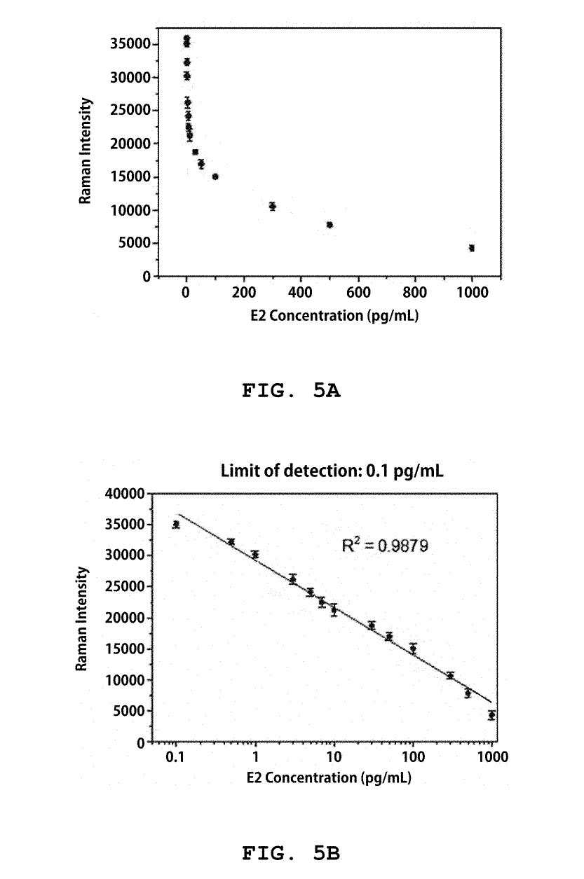

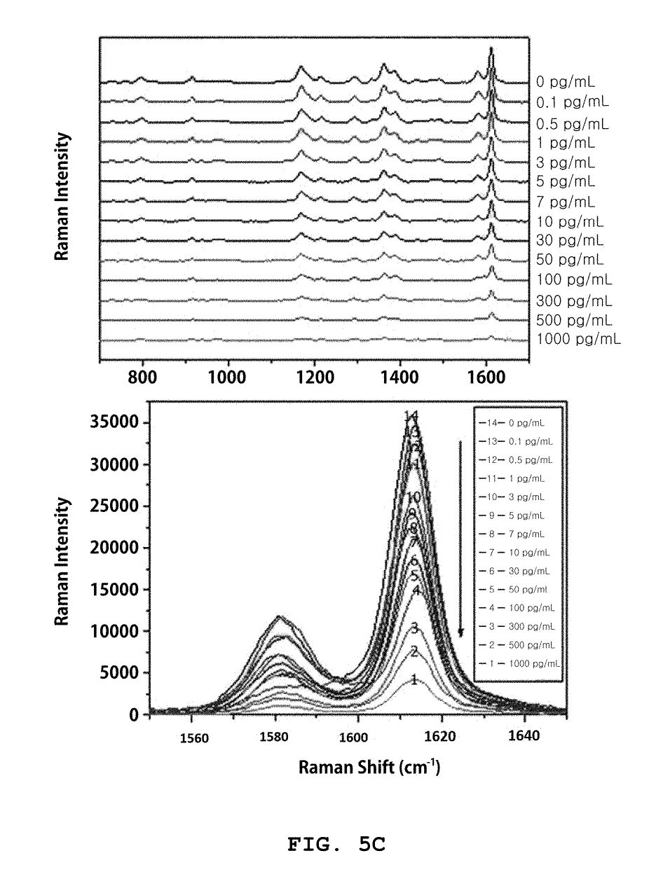

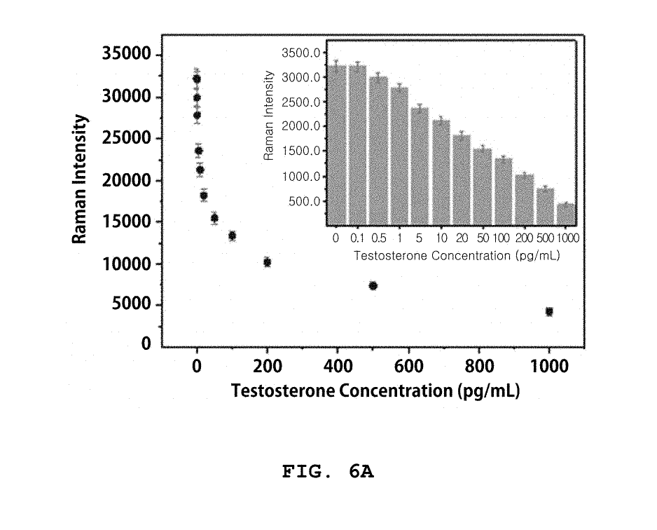

The Raman signals were obtained as shown in FIGS. 5a-5c. The limit of detection was 0.1 pg/mL.

3-2: Detection of Testosterone

First, blood containing testosterone was prepared as a sample solution. Then, 25 μL of the magnetic particles in which the secondary antibodies and the primary antibodies (anti-testosterone antibody) are immobilized, which was synthesized in Example 2, and 50 μL of the gold nanoprobes prepared in Example 1 were added at the same time to 25 μL of the sample solution. A total of 90 minutes was spent for the detection.

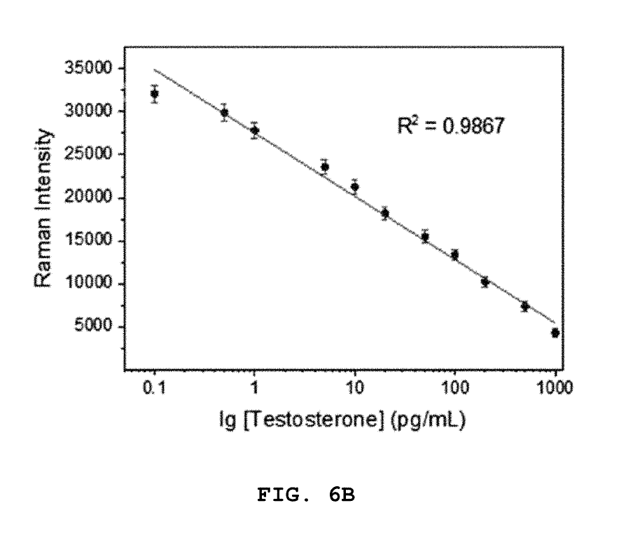

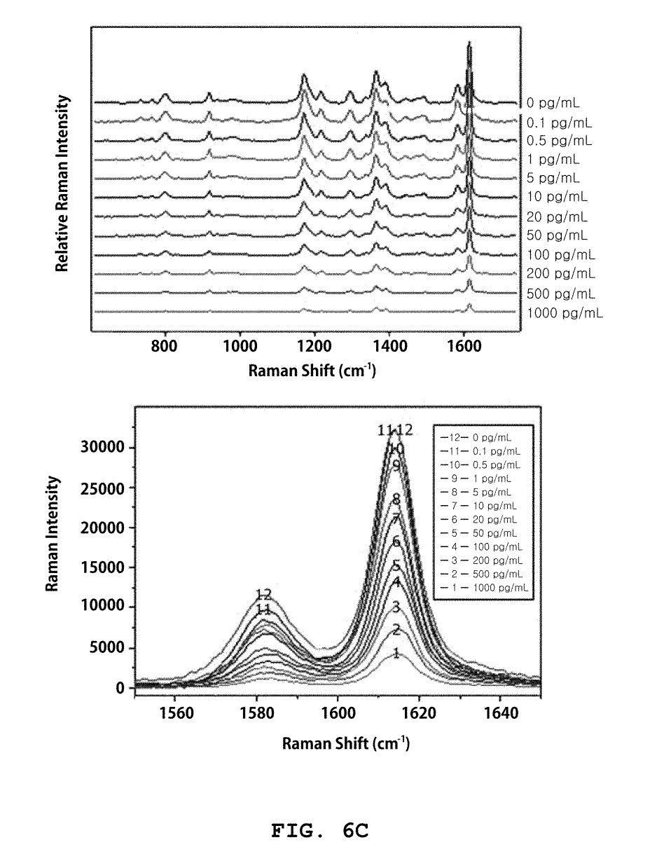

Raman analysis was performed in the same manner as described in Example 3-1. The Raman signals were obtained as shown in FIGS. 6a-6c. The limit of detection was 0.1 pg/mL.

### COMPARATIVE EXAMPLE

### Comparison with ELISA Analysis

1: ELISA Analysis for Detection of Estradiol

Enzyme immunoassay (ELISA) is a commonly employed diagnostic method based on the color change of blood to which an antigen is added due to enzymatic action. To assess the detection sensitivity of the sex hormone analyzing technique based on surface-enhanced Raman spectroscopy according to the present disclosure, it was compared with an analysis result using the Abnova estradiol detection kit (based on ELISA). This diagnosis method performs quantitative analysis through competitive reaction of estradiol in an analyte sample with estradiol labeled with a luminescence-inducing material. After attaching antibodies that can immobilize estradiol onto a plate and adding the analyte sample, the antibodies conjugated with the luminescence-inducing material were allowed to be bound to the immobilized antigens. Diagnosis was made by measuring color change depending on the content of the luminescence-inducing material. The substances used in the analysis are described in Table 2.

TABLE 2

| Component | Amount | |
|---|---|---|
| Goat Anti-Rabbit IgG-coated microtiter wells | 96 | wells |
| Estradiol Reference Standards: 0, 10, 30, 100, | 0.5 | ml each |
| 300, and 1000 pg/ml. Liquid, ready to use. | | |
| Rabbit Anti-Estradiol Reagent (pink color) | 7 | ml |
| Estradiol-HRP Conjugate Reagent (blue color) | 12 | ml |
| Estradiol Control 1, Liquid, Ready to use | 0.5 | ml |
| Estradiol Control 2, Liquid, Ready to use | 0.5 | ml |
| TMB Reagent (One-Step) | 11 | ml |
| Stop Solution (1N HCl) | 11 | ml |

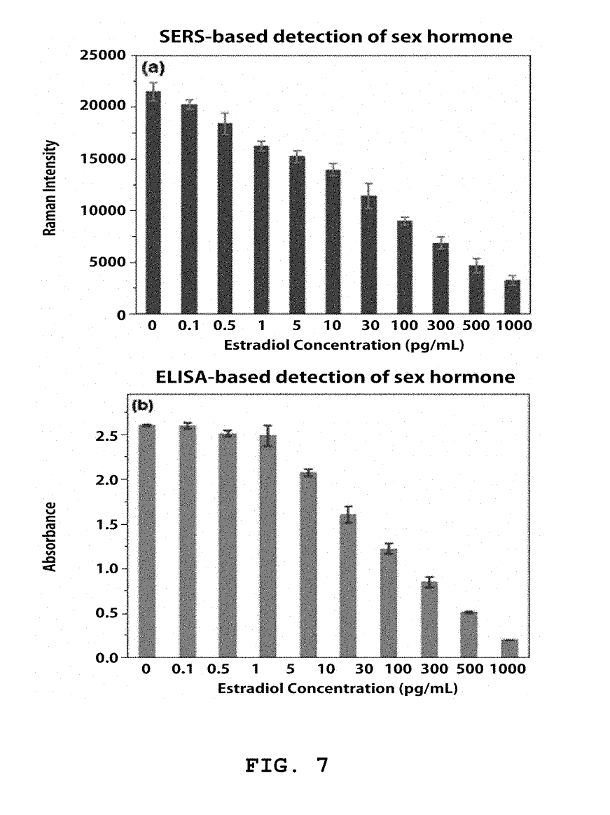

FIG. 7 compares the results of detecting the sex hormone based on surface-enhanced Raman scattering (a) and ELISA analysis (b). To compare the two detection methods, for the SERS-based detection method according to the present disclosure, the detectable range was 0.1-1,000 pg/mL and the detection limit was 0.1 pg/mL. In contrast, for the ELISA analysis method, the detectable range was 5-1,000 pg/mL and the detection limit was 5 pg/mL.

2: ELISA Analysis for Detection of Testosterone

The substances used in the ELISA analysis are described in Table 3.

TABLE 3

| Component | Amount | |
|---|---|---|
| Testosterone-Coated Wells: microtiter wells coated with | 1 | plate, |
| testosterone-BSA conjugates | 96 | wells |
| Reference Standard Set: Contains 0, 1, 5, 10, 25, 50, | 0.5 | ml/vial |
| 75 and 100 ng/ml testosterone, liquid, ready to use. | | |
| Mouse Anti-Testosterone Reagent: Contains mouse | 7 | ml |
| anti-testosterone in bovine serum albumin (BSA) buffer | | |
| Goat Anti-Mouse IgG HRP Conjugate Reagent: Contains | 12 | ml |
| goat anti-mouse IgG conjugated to HRP | | |
| Washing Buffer (PBS-Tween 20, 0.1%, v/v) | 12 | ml |
| TMB Reagent: Contains 3,3′,5,5′-TMB stabilized in buffer | 11 | ml |
| solution | | |
| Stop Solution: Diluted hydrochloric acid (1N HCl) | 11 | ml |

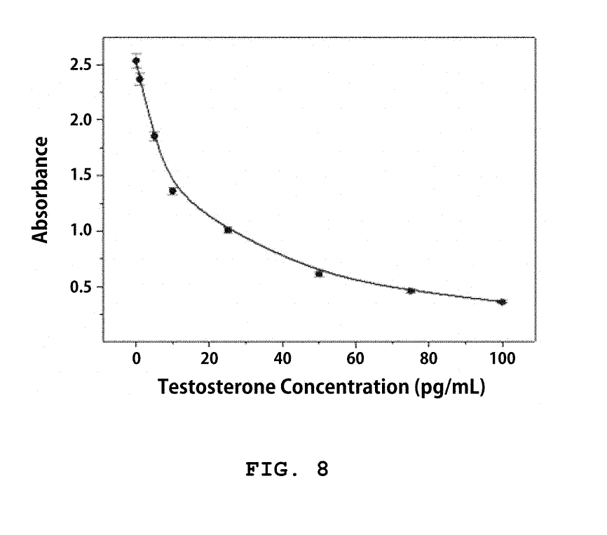

FIG. 8 compares the result of detecting testosterone by ELISA analysis. For the SERS-based detection method according to the present disclosure, the detectable range was 0.1-1,000 pg/mL and the detection limit was 0.1 pg/mL. In contrast, for the ELISA analysis method, the detectable range was 1-100 pg/mL and the detection limit was 0.18 ng/mL (=180 pg/mL).

From these results, it can be seen that the SERS-based detection method according to the present disclosure allows analysis of the sample at concentration ranges of 0.1-5 pg/mL, which is impossible with the ELISA analysis method. In particular, the present disclosure meets the requirement of detection of the sex hormone at low concentrations of below 10 pg/mL, which is necessary for the diagnosis of precocious puberty. In contrast, detection of the sex hormone at low concentrations of below 10 pg/mL is impossible with the ELISA analysis method. Accordingly, it can be seen that the SERS-based detection method according to the present disclosure is an analysis method capable of detecting the sex hormone with high sensitivity, which is necessary for the diagnosis of precocious puberty.

### EXAMPLE 4

### Evaluation of Clinical Applicability

30 blood samples were analyzed by the Architect's estradiol assay (automated assay). The Architect's estradiol detection method is an immunoassay using a chemiluminescent material and is capable of quantitative analysis based on chemiluminescence signals depending on the amount of the sex hormone present in blood. The detectable range of this method is 10-1000 pg/mL and the detection limit is 10 pg/mL. Accordingly, analysis is impossible for samples at concentrations below 10 pg/mL. Table 4 shows the result of testing the concentration of estradiol in the 30 blood samples using the Architect's assay system. And, the result of analyzing the same blood samples using the SERS-based detection method according to the present disclosure is compared in Table 5. From the analysis result given in Table 5, it can be seen that very significantly results are attained for the 30 blood samples with the SERS-based detection method as compared to the Architect's estradiol detection method. In particular, it can be seen that even the samples with estradiol concentrations lower than 10 pg/mL could be analyzed accurately, which was impossible with the Architect's assay system. Through this, the clinical applicability of the SERS-based detection method according to the present disclosure was verified. In particular, the method is very superior in analyzing blood samples with sex hormone concentrations lower than 10 pg/mL as compared to the existing analysis equipment. Accordingly, it can be seen that the SERS-based detection method according to the present disclosure is a very suitable method for diagnosis of precocious puberty through detection of sex hormones in blood with high sensitivity.

TABLE 4

| Number | Gender | Age | E2conc. (pg/mL) |
|---|---|---|---|
| 101 | F | 13 | 39 |
| 102 | F | 10 | 27 |
| 103 | F | 8 | 15 |
| 104 | F | 8 | <10 |
| 105 | F | 12 | 56 |
| 106 | F | 8 | 18 |
| 107 | F | 8 | <10 |
| 108 | F | 8 | <10 |
| 109 | F | 8 | <10 |
| 110 | F | 8 | 14 |
| 111 | F | 8 | <10 |
| 112 | F | 9 | <10 |
| 113 | F | 13 | 29 |
| 114 | F | 10 | <10 |
| 115 | F | 8 | 17 |
| 116 | M | 15 | 22 |
| 117 | F | 7 | <10 |
| 118 | F | 8 | <10 |
| 119 | F | 10 | <10 |
| 120 | F | 10 | 89 |
| 121 | F | 9 | <10 |
| 122 | F | 9 | 12 |
| 123 | F | 9 | 17 |
| 124 | M | 10 | 11 |
| 125 | F | 8 | 16 |
| 126 | F | 10 | 19 |
| 127 | F | 8 | 15 |
| 128 | F | 8 | 12 |
| 129 | F | 9 | 13 |
| 130 | F | 9 | 26 |

TABLE 5

| | | CMI Assay | SERS Assay |
|---|---|---|---|
| Grade | Sample No. | (pg/mL) | (pg/mL) |
| Negative | 104 | <10 | 6.2 |
| | 107 | <10 | 5.3 |
| | 108 | <10 | 2.4 |
| | 109 | <10 | 7.9 |
| | 111 | <10 | 4.1 |
| | 112 | <10 | 8.8 |
| | 114 | <10 | 8.6 |
| | 117 | <10 | 9.9 |
| | 118 | <10 | 6.7 |
| | 119 | <10 | 9.6 |
| | 121 | <10 | 3.9 |
| Low Positive | 103 | 15 | 18.8 |
| | 106 | 18 | 21.2 |
| | 110 | 14 | 19.1 |
| | 115 | 17 | 25.3 |
| | 122 | 12 | 16.7 |
| | 123 | 17 | 20.1 |
| | 124 | 11 | 15.9 |
| | 125 | 16 | 24.9 |
| | 126 | 19 | 22.7 |
| | 127 | 15 | 16.1 |
| | 128 | 12 | 13.9 |
| | 129 | 13 | 17.1 |
| Positive | 101 | 39 | 43.8 |
| | 102 | 27 | 31 |
| | 105 | 56 | 52.9 |
| | 113 | 29 | 33.2 |
| | 116 | 22 | 24.4 |
| | 120 | 89 | 98.3 |
| | 130 | 26 | 27.1 |

1. A reagent kit for detecting a sex hormone, which comprises:
a first reagent comprising a metal nanoprobe in which a sex hormone and a Raman reporter are immobilized; and
a second reagent comprising a magnetic particle in which an antibody for detecting the sex hormone is immobilized.

2. The reagent kit for detecting a sex hormone according to claim 1, wherein the antibody for detecting the sex hormone comprises a primary antibody and a secondary antibody, the secondary antibody is immobilized on the magnetic particle and the primary antibody binds to the secondary antibody and reacts with the sex hormone.

3. The reagent kit for detecting a sex hormone according to claim 1, wherein the sex hormone is estrogen or testosterone.

4. The reagent kit for detecting a sex hormone according to claim 3, wherein the estrogen is one or more selected from a group consisting of estradiol, estrone and estriol.

5. The reagent kit for detecting a sex hormone according to claim 1, wherein the detectable concentration of the sex hormone is 0.1-1,000 pg/mL.

6. The reagent kit for detecting a sex hormone according to claim 1, wherein the limit of detection of the sex hormone is 0.1 pg/mL.

7. The reagent kit for detecting a sex hormone according to claim 1, wherein the detection time of the sex hormone is 2 hours or shorter.

8. A SERS-based method for detecting a sex hormone comprising the steps of:
preparing a sample solution comprising a sex hormone;
preparing a metal nanoprobe in which the sex hormone is bound to a Raman reporter;
preparing a magnetic particle in which a primary antibody and a secondary antibody for detecting the sex hormone are immobilized;
adding the metal nanoprobe and the magnetic particle in which the primary antibody and the secondary antibody are immobilized to the sample solution at the same time;
forming an immunocomplex with the magnetic particle through a competitive immunoreaction of each of the sex hormone in the sample solution and the sex hormone of the metal nanoprobe with the primary antibody immobilized in the magnetic particle;
separating the magnetic particle in which the immunocomplex is formed using magnetism;
irradiating a laser light to the separated magnetic particle; and
detecting the sex hormone by measuring a surface-enhanced Raman scattering (SERS) signal after the irradiation of the laser light.

9. The SERS-based method for detecting a sex hormone according to claim 8, wherein, in the step of forming the immunocomplex,
the intensity of the SERS signal is decreased as the concentration of the sex hormone in the sample solution is higher because the amount of the metal nanoprobe forming the immunocomplex with the magnetic particle is decreased; or
the intensity of the SERS signal is increased as the concentration of the sex hormone in the sample solution is lower because the amount of the metal nanoprobe forming the immunocomplex with the magnetic particle is increased.

10. The SERS-based method for detecting a sex hormone according to claim 8, wherein the sex hormone is estrogen or testosterone.

11. The SERS-based method for detecting a sex hormone according to claim 10, wherein the estrogen is one or more selected from a group consisting of estradiol, estrone and estriol.

12. The SERS-based method for detecting a sex hormone according to claim 8, wherein the sample solution is selected from a group consisting of a tissue extract, a cell lysate, a whole blood, a blood plasma, a blood serum, a saliva, an ocular fluid, a cerebrospinal fluid, a sweat, a urine, a milk, an ascitic fluid, a synovial fluid, a peritoneal fluid and a dried blood spot.

13. The SERS-based method for detecting a sex hormone according to claim 8, wherein the secondary antibody is an anti-mouse antibody immobilized on the magnetic particle and the primary antibody is an anti-sex hormone antibody which binds to the secondary antibody and specifically immunoreacts with the sex hormone.

14. The SERS-based method for detecting a sex hormone according to claim 8, wherein the detectable concentration of the sex hormone is 0.1-1,000 pg/mL.

15. The SERS-based method for detecting a sex hormone according to claim 8, wherein the limit of detection of the sex hormone is 0.1 pg/mL.

16. The SERS-based method for detecting a sex hormone according to claim 8, wherein the detection time of the sex hormone is 2 hours or shorter.

17. A method for diagnosis of precocious puberty of a subject comprising the steps of:
extracting a sample solution from a subject;
detecting a sex hormone for the extracted sample solution by the method for detecting a sex hormone according to claim 8; and
determining the concentration of the detected sex hormone.

18. The method according to claim 17, wherein the concentration of the detected sex hormone is 0.1-1000 pg/mL.

19. The method according to claim 17, which further comprises, after the step of determining the concentration of the detected sex hormone, a step of diagnosing as precocious puberty when the concentration of the detected sex hormone is higher than 10 pg/mL.
